(19) United States
(12) Patent Application Publication
(10) Pub. No.: US 2022/0071796 A1
(43) Pub. Date: Mar. 10, 2022

(54) Device for Pumping Air

(71) Applicants: Yun-Wen Lee, Taipei (TW); Chih-Lung Chein, Taipei (TW)

(72) Inventors: Yun-Wen Lee, Taipei (TW); Chih-Lung Chein, Taipei (TW)

(21) Appl. No.: 17/015,160

(22) Filed: Sep. 9, 2020

(51) Int. Cl. A61F 5/41; A61B 5/024; A61B 5/00

(52) U.S. Cl. CPC A61F 5/41; A61F 2005/415; A61B 5/681; A61B 5/02438

(57) ABSTRACT

A device is provided for pumping air. The device comprises a detection unit and an air-supply unit. The air-supply unit comprises an air-pumping module and a balloon. The air-pumping module connects to the balloon; the air-pumping module electrically connects to the detection unit; and the balloon has a placement area. Thereby, a user puts a penis in the balloon, where the detection unit is coordinated with the heartbeat of the user to drive the air-pumping module for continuously processing operations of deflating and inflating to the balloon by the air-pumping module to massage the penis by the balloon. Thus, the penis is pressured up with the blood of the user himself to unblock peripheral vessels at the penis for restoring physiological function of the penis.

### TECHNICAL FIELD OF THE INVENTION

The present invention relates to a device for pumping air; more particularly, to pressuring up a penis with the blood of a user himself for restoring physiological function of the penis.

### DESCRIPTION OF THE RELATED ARTS

Human body organs need the smooth flow of blood maintained by vessels to function properly. The vessels are rooted deep down into each body organ and tissue, like brain, heart, liver, skin, limb, etc. The vessels are pipes of providing blood, cells, and various hormones to nerves, muscles, bones, and other tissues with enough blood and nutrients for maintaining the proper functioning of human organs. However, where blood vessels are normally multiplied in number and not deformed for the blood to flow in an unobstructed way, poor peripheral-blood circulation would be the result if the vessels are deformed or led to blockage due to illness. Thereby, in addition to causing cold extremities easily, human body would show a lot more uncomfortable symptoms. To a man, if the peripheral-blood circulation is poor, the penis may be easily led to inadequate congestion causing sexual dysfunctions of premature ejaculation, impotence, and so on.

### SUMMARY OF THE INVENTION

The main purpose of the present invention is to put a penis in a balloon by a user with a detection unit coordinated with the heartbeat of the user to drive an air-pumping module for continuously processing operations of deflating and inflating to the balloon by the air-pumping module to massage the penis by the balloon, where, thereby, the penis is pressured up with the blood of the user himself to unblock peripheral vessels at the penis for restoring physiological function of the penis.

To achieve the above purpose, the present invention is a device for pumping air, comprising a detection unit and an air-supply unit, where the air-supply unit comprises an air-pumping module and a balloon; the air-pumping module connects to the balloon; the air-pumping module electrically connects to the detection unit; and the balloon has a placement area. Accordingly, a novel device for pumping air is obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the view showing the preferred embodiment according to the present invention; and

FIG. 2 is the view showing the state-of-use.

### DESCRIPTION OF THE PREFERRED EMBODIMENT

The following description of the preferred embodiment is provided to understand the features and the structures of the present invention.

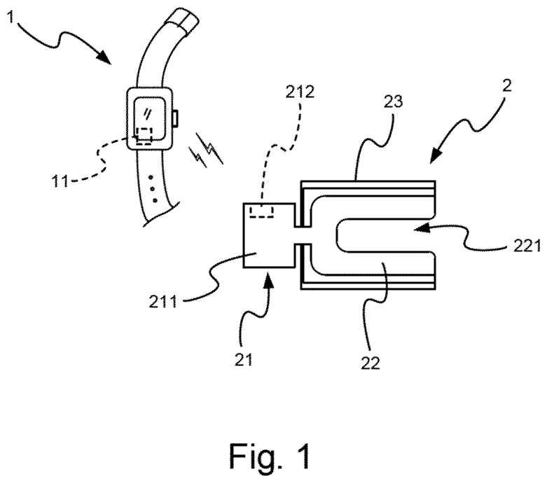

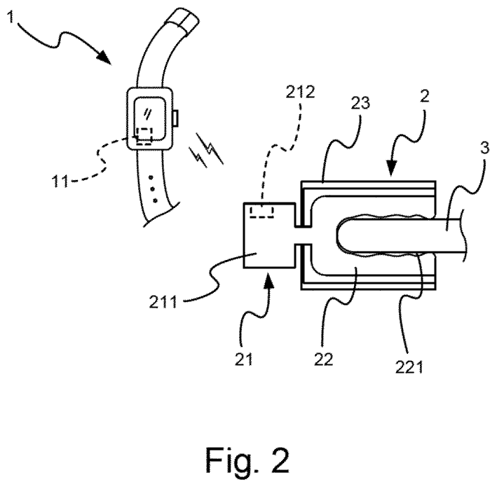

Please refer to FIG. 1 and FIG. 2, which are a view showing a preferred embodiment according to the present invention; and a view showing a state-of-use. As shown in the figures, the present invention is a device for pumping air, comprising a detection unit 1 and an air-supply unit.

The detection unit 1 has a heart-rate sensor 11 and is a smart watch or a smart bracelet.

The air-supply unit 2 comprises an air-pumping module 21 and a balloon 22. The air-pumping module 21 connects to the balloon 22; the air-pumping module 21 electrically connects to the heart-rate sensor 11 of the detection unit 1; and the balloon 22 has a placement area 221.

On using the present invention, a user wears the detection unit 1 and the user puts a penis 3 in the placement area 221 of the balloon 22. The heart-rate sensor 11 of the detection unit 1 is coordinated with the heart rhythm of the user to drive the air-pumping module of the air-supply unit 2 for continuously processing operations of deflating and inflating to the balloon 22 by the air-pumping module 21. For example: After two heartbeats, the air-pumping module 21 inflates the balloon 22 and then deflates it, or, the air-pumping module 21 inflates the balloon 22 during cardiac systole and the air-pumping module 21 deflates the balloon 22 during cardiac diastole. The balloon 2 massages the penis 3 of the user to thereby pressure the penis 3 with the blood of the user himself for unblocking peripheral vessels at the penis 3 to restore physiological function of the penis 3.

In a state-of-use, the air-pumping module 21 comprises a pump 211 and a receiver 212. The pump 211 connects to the balloon 22; and the receiver 212 electrically connects to the detection unit 1 in a wired way or a wireless way. Hence, the detection unit 1 electrically connects to the receiver 212 of the air-pumping module 21 to process handshaking with the air-supply unit 2 by the detection unit 1, so that the heart-rate sensor 11 is coordinated with the heart rhythm of the user to drive the air-pumping module 21 of the air-supply unit 2 for continuously processing operations of inflating and deflating to the balloon 22 by the pump 211 of the air-pumping module 21. The balloon 2 massages the penis 3 of the user to thereby pressure the penis 3 with the blood of the user himself for unblocking peripheral vessels at the penis 3 to restore physiological function of the penis 3.

In a state-of-use, the balloon 22 is of a soft plastic material. Thus, when the user puts the penis 3 in the placement area 221 of the balloon 22 with the penis 3 of the user massaged by the balloon 2, a better comfort is obtained to avoid injuring the penis 3.

In a state-of-use, the air-supply unit 2 further comprises a carrier 23, where the balloon 22 is set in the carrier 23 and the carrier 23 is a hollow cylinder. Consequently, not only the carrier 23 limits the balloon 22 to a predetermined shape, but also the user holds the carrier 23 easily with his hand to steadily put the penis 3 in the placement area 221 of the balloon 22 for achieving easy operation, where the balloon 2 is inflated/deflated at a rate of 10˜160 times per minute.

To sum up, the present invention is a device for pumping air, where a penis is put into a balloon by a user with a detection unit coordinated with the heartbeat of the user to drive an air-pumping module for continuously processing operations of deflating and inflating to the balloon by the air-pumping module to massage the penis by the balloon, where, thereby, the penis is pressured up with the blood of the user himself to unblock peripheral vessels at the penis for restoring physiological function of the penis.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A device for pumping air, comprising
a detection unit; and
an air-supply unit, comprising an air-pumping module and a balloon,
wherein said air-pumping module connects to said balloon; said air-pumping module electrically connects to said detection unit; and said balloon has a placement area.

2. The device according to claim 1,
wherein said detection unit has a heart-rate sensor and is a smart watch or a smart bracelet.

3. The device according to claim 1,
wherein said air-pumping module comprises a pump and a receiver; said pump connects to said balloon; and said receiver electrically connects to said detection unit in a way selected from a group consisting of a wired way and a wireless way.

4. The device according to claim 1,
wherein said balloon is of a soft plastic material.

5. The device according to claim 1,
wherein said air-supply unit further comprises a carrier and said balloon is deposited in said carrier.

6. The device according to claim 1,
wherein said balloon is inflated/deflated at a rate of 1˜160 times per minute.
